(19) United States
(12) Patent Application Publication
(10) Pub. No.: US 2021/0355452 A1
(43) Pub. Date: Nov. 18, 2021

(54) PURIFICATION METHOD FOR VACCINE VIRUS USING AFFINITY CHROMATOGRAPHY

(71) Applicant: HK INNO.N CORPORATION, Seoul (KR)

(72) Inventors: Jaelim YU, Icheon-si (KR); Jina CHAE, Yongin-si (KR); Eun Ju JUNG, Yongin-si (KR); Dong Eok LEE, Seoul (KR)

(21) Appl. No.: 17/291,210

(22) PCT Filed: Dec. 19, 2019
(86) PCT No.: PCT/KR2019/018101

§ 371 Date: May 4, 2021

(30) Foreign Application Priority Data
Dec. 20, 2018 (KR) 10-2018-0166428

(51) Int. Cl. C12N 7/00; B01D 15/42; B01D 15/20; B01D 15/38

(52) U.S. Cl. CPC C12N 7/00; B01D 15/426; C12N 2770/00034; B01D 15/3804; C12N 2770/00051; B01D 15/203

(57) ABSTRACT

The present disclosure relates to separation and purification methods for a vaccine virus using affinity chromatography, and more particularly, to a purification method for a virus capable of obtaining a vaccine virus with a high purity and a high yield using affinity chromatography containing a vaccine virus-affinity resin.

### TECHNICAL FIELD

The present disclosure relates to separation and purification methods for a vaccine virus using affinity chromatography, and more particularly, to separation and purification methods for a virus capable of obtaining a vaccine virus with a high purity and a high yield using affinity chromatography containing a virus-affinity resin.

### BACKGROUND ART

In vaccine viruses cultured using cells derived from species other than humans as host cells, it is necessary to remove host-derived materials. In order to remove the host-derived materials, in the related art, a sugar density gradient centrifugation method, size-exclusion chromatography, or ion-exchange chromatography has been used. As methods frequently used in virus purification, these methods are used more than affinity chromatography because the methods are easily applicable regardless of the type of virus.

The sugar density gradient centrifugation method is a method of purifying viruses using a density difference made using sugar, and as the most traditional and oldest method, it is the method which is used most in the initial stages of studies, as it does not require separate process studies. In order to apply the method to an industrial production stage, expensive equipment is additionally required, and a process such as a dialysis process or size-exclusion chromatography for removing sugar needs to be added, and thus there is a disadvantage in that the total processing time is long. It was also reported in a study that viscosity and high osmotic pressure of the sugar affects infective proteins of the virus to reduce the overall virus yield of the process (Peng H H et al. (2006) Anal Biochem, 354(1):140-147).

The size-exclusion chromatography method is a method without effects due to protein modification or osmotic pressure, and in the prior art (CN101695570B, CN101780278B), it is disclosed that an inactivated vaccine for hand-foot-and-mouth disease is prepared using the size-exclusion chromatography method. However, in the size-exclusion chromatography method, since an excessive concentration process is involved as a pre-treatment process, there is a disadvantage in that the viral structure is broken due to the concentration process, or the yield is reduced due to addition of a process. Further, in the size-exclusion chromatography method, since there is a limit to the scale-up, application thereof is relatively easy in study stages, but there is a limit to its application at a scale for industrial mass production.

Studies have been conducted using ion-exchange chromatography, which may be used regardless of the volume of a virus sample (CN101695570B, Ashok Raj Kattur Venkatachalam et al. (2014) Virology Journal, 11:99). Most studies were conducted by a method of adsorbing a virus to a resin having charges, such as DEAE, and then eluting the adsorbed virus with a buffer having a high salt concentration. However, in order to use the ion-exchange chromatography, a dialysis process is required to lower the salt concentration of the sample, and there is a disadvantage in that the yield is reduced due to addition of a process. In addition, since the virus consists of various types of proteins rather than a single protein, the virus has various charges, and thus a process study for maintain a virus elution condition is required. In addition, there is a disadvantage in that impurities having similar charges to the virus may be eluted together.

### DISCLOSURE OF INVENTION

### Technical Problem

With this background, the present inventors made an effort to find a method of purifying a vaccine virus with a high purity and a high yield, and as a result, they found a purification method capable of obtaining a vaccine virus with a high purity and a high yield when affinity chromatography was used, thereby completing the present disclosure.

### Solution to Problem

An aspect of the present disclosure provides a purification method for a vaccine virus comprising: (a) loading a sample containing a vaccine virus on an affinity chromatography column containing a virus-affinity resin; (b) washing the affinity chromatography column with a washing solution; and (c) recovering a desired vaccine virus from the affinity chromatography column using an elution solution.

Another aspect of the present disclosure provides a vaccine virus purified according to the purification method.

### Advantageous Effects of Invention

According to the purification method of the present disclosure, while most impurities other than a desired vaccine virus are removed, the vaccine virus may be purified with a high purity and a high yield suitable for mass production.

### BRIEF DESCRIPTION OF DRAWINGS

FIG. 1 illustrates a procedure of performing a purification method of the present disclosure.

FIGS. 2 and 3 illustrate a result of purifying a vaccine virus using a Capto™ DeVirS resin containing dextran sulfate.

FIGS. 4 and 5 illustrate a result of purifying a vaccine virus using a HiTrap Heparin resin containing heparin.

FIGS. 6 and 7 illustrate a result of purifying a vaccine virus using a Fractogel DEAE resin.

FIGS. 8 and 9 illustrate a result of purifying a vaccine virus using a Fractogel TMAE resin.

FIGS. 10 and 11 illustrate a result of purifying a vaccine virus using a CIM DEAE resin.

### BEST MODE FOR CARRYING OUT THE INVENTION

Hereinafter, the present disclosure will be described in more detail.

Meanwhile, each description and embodiment disclosed in the present disclosure can also be applied to each of the other descriptions and embodiments. That is, all combinations of the various components disclosed in the present disclosure belong to the scope of the present disclosure. In addition, the scope of the present disclosure may not be limited by the specific description below.

Further, those skilled in the art may recognize or determine a plurality of equivalents to specific embodiments of the present disclosure described in the present disclosure by using only general experimentation. In addition, such equivalents are intended to be included in the present disclosure.

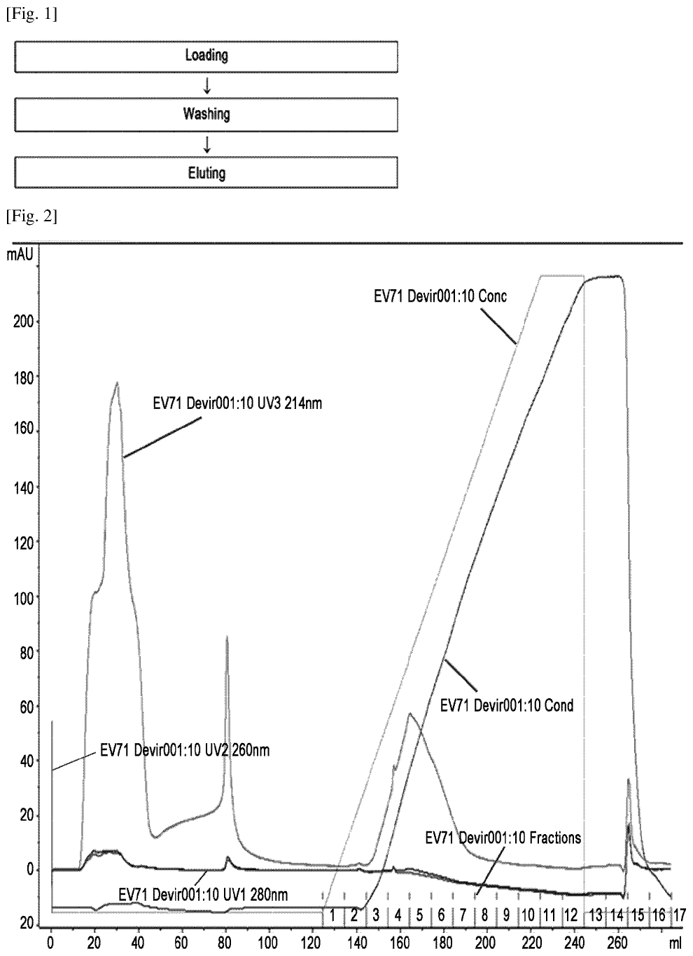

FIG. 1 illustrates an example of a procedure of performing a purification method of the present disclosure.

Referring to FIG. 1, an aspect of the present disclosure provides a purification method for a vaccine virus comprising: (a) loading a sample containing a vaccine virus on an affinity chromatography column containing a virus-affinity resin; (b) washing the affinity chromatography column with a washing solution; and (c) recovering a desired vaccine virus from the affinity chromatography column using an elution solution.

Each step of the purification method for the vaccine virus will be described in detail as follows. First, step (a) is a step of loading the sample containing the vaccine virus on the affinity chromatography column containing the virus-affinity resin.

As long as the sample containing the vaccine virus contains a vaccine virus, there is no limitation to materials and manufacturing methods. Specifically, the sample containing the vaccine virus may include an enterovirus, but is not limited thereto. The sample may be prepared from host cells other than human-derived cells, but is not limited thereto.

The “affinity chromatography” used in the present disclosure refers to a chro-matography method using a material that binds to a specific protein with affinity. The material binding to the specific protein with affinity is a material in which a function group is conjugated to a polymeric material, and binds to a material having affinity which is dissolved in a polar or non-polar solution.

For the purpose of the present disclosure, the affinity chromatography may be affinity chromatography containing a vaccine virus-affinity resin. Specifically, the chromatography may be performed using a resin capable of specifically binding to the vaccine virus protein. As an example, the vaccine virus-affinity resin may include at least one selected from the group consisting of dextran sulfate, heparin, and mixtures thereof. For example, the vaccine virus-affinity resin includes Capto™ DeVirS (GE Healthcare) and HiTrap Heparin (GE Healthcare), but is not limited thereto, and any resin capable of specifically binding to the vaccine virus protein is possible.

As an example, the Capto™ DeVirS resin contains dextran sulfate, the HiTrap Heparin resin contains heparin, and the resins may specifically bind to the vaccine virus protein.

In one embodiment, before loading the sample containing the vaccine virus in step (a), a column may be equilibrated with an equilibrium solution of pH 7.5 to pH 8.0. Specifically, the equilibrium solution may include at least one salt selected from the group consisting of sodium phosphate, sodium chloride, Tris-HCl, 2-(N-morpholino)ethanesulfonic acid (MES), 3-morpholinopropane-1-sulfonic acid (MOPS), PIPES, potassium phosphate, potassium chloride, and 2-[4-(2-hydroxyethyl)piperazin-1-yl]ethanesulfonic acid (HEPES), but is not limited thereto.

In one embodiment, the method may further include ion-exchange chromatography, concentration, and/or dialysis before step (a). This step is to increase the purity of the sample by removing primary impurities in the sample containing the vaccine virus. Specifically, before step (a), the sample containing the vaccine virus is concentrated and dialyzed, and after pre-performing purification by ion-exchange chromatography, the sample containing the vaccine virus may be loaded on the affinity chromatography column using the affinity resin. Any operation for removing the primary impurities which do not bind to the affinity resin and enhancing the purity of the sample may be applied without limitation.

In one embodiment, the purification method for the vaccine virus using the affinity chromatography may be characterized in that a separate concentration or dialysis process is not performed before the affinity chromatography. In this case, while the process is simple, it is possible to obtain a result with a high yield and a high purity.

In the purification method for the vaccine virus, step (b) is a step of applying a washing solution to the chromatography column on which the sample is loaded, as a step of washing the sample with the washing solution. [41] The washing solution may have a range of pH 7.5 to pH 8.0. Specifically, the washing solution may include at least one salt selected from the group consisting of sodium phosphate, sodium chloride, Tris, 2-(N-morpholino)ethanesulfonic acid (MES), 3-morpholinopropane-1-sulfonic acid (MOPS), PIPES, potassium phosphate, potassium chloride, and 2-[4-(2-hydroxyethyl)piperazin-1-yl]ethanesulfonic acid (HEPES), but is not limited thereto.

For the purpose of the present disclosure, in step (b), impurities which non-specifically bind to the vaccine virus-affinity resin may be removed by the washing solution.

In one embodiment, the purification method may further include a step of discharging impurities without affinity with the resin with the equilibrium solution after step (a) or (b). The step may be specifically performed at least once, but generally, may be performed without limitation until equilibrium is achieved.

In one embodiment, the purification method may further include a step of performing re-equilibration with a re-equilibrium solution after step (a) or (b). The re-equilibrium solution does not react with anything between the washing step and the eluting step, flows under the same conditions as the equilibrium solution in step (a) from which the desired vaccine virus is not eluted, and then flows again before the elution solution flows to serve as a bridge between the washing solution and the elution solution.

Specifically, the re-equilibrium solution may have a range of pH 7.5 to pH 8.0.

Specifically, the re-equilibrium solution may include at least one salt selected from the group consisting of sodium phosphate, sodium chloride, Tris-HCl, 2-(N-morpholino)ethanesulfonic acid (MES), 3-morpholinopropane-1-sulfonic acid (MOPS), PIPES, potassium phosphate, potassium chloride, and 2-[4-(2-hydroxyethyl)piperazin-1-yl]ethanesulfonic acid (HEPES), but is not limited thereto.

In the purification method for the virus, step (c) is a step of recovering the desired vaccine virus from the affinity chromatography column using the elution solution.

The elution solution may have a range of pH 7.5 to pH 8.0. Specifically, the elution solution may include at least one salt selected from the group consisting of sodium phosphate, sodium chloride, Tris-HCl, 2-(N-morpholino)ethanesulfonic acid (MES), 3-morpholinopropane-1-sulfonic acid (MOPS), PIPES, potassium phosphate, potassium chloride, and 2-[4-(2-hydroxyethyl)piperazin-1-yl]ethanesulfonic acid (HEPES), but is not limited thereto.

In addition, the elution solution may contain 0.1 M to 0.5 M sodium chloride, but the salts which may separate the desired vaccine virus from the affinity chro-matography column may be used without limitation of the concentration.

The desired vaccine virus separated using the purification method of the present disclosure may have a purity of 88% or higher, and specifically, a purity of 90% or higher, 91% or higher, 92% or higher, 93% or higher, 94% or higher, 95% or higher, 96% or higher, 97% or higher, or 98% or higher, but is not limited thereto. The term “purity” means a pure vaccine virus from which the impurities are removed, and as an example, if the purity is 92%, the remaining 8% means impurities. Additionally, the purity may simply represent the purity of the material separated from the eluted solution, but the final purity % may vary according to what % the purity of the loaded sample is.

The term “impurity” is any material other than the desired vaccine virus, and for example, may include a host-derived DNA, a host-derived protein, an endotoxin, etc., but is not limited thereto.

Further, the purity of the vaccine virus may be analyzed by an enzyme-linked immunosorbent assay (ELISA) method specifically provided to measure host-derived impurities from a total protein amount of the elution solution, but is not limited thereto, and of course, the purity of the vaccine virus may be analyzed using CEX-HPLC, SEC-HPLC, etc.

In the present disclosure, the virus is preferably an enterovirus, but is not limited thereto.

Further, the vaccine virus purified by the purification method of the present disclosure may be used as a vaccine or immunogenic composition, but is not limited thereto.

Another aspect of the present disclosure provides a vaccine virus purified according to the purification method. The vaccine virus may be used as a vaccine or immunogenic composition, but is not limited thereto.

Mode for the Invention

Hereinafter, preferred Examples are proposed to assist understanding of the present disclosure. However, the following Examples are merely provided so that the present disclosure may be more easily understood, and contents of the present disclosure are not limited by Examples.

### EXAMPLE 1

### Purification using Capto™ DeVirS Resin Containing Dextran Sulfate

In Example 1, a purification yield for a vaccine virus and an impurity removal rate were confirmed using a Capto™ DeVirS resin containing a dextran sulfate ligand.

A 20 mM sodium phosphate pH 7.5 buffer was used as an equilibrium solution and a washing solution (0 M sodium chloride), and an elution solution was prepared and used with a pH 7.5 buffer in which sodium chloride would reach 2 M in the equilibrium solution.

First, a vaccine virus-containing sample containing an enterovirus was loaded on a column, and then washing was performed by flowing with the washing solution. Next, an elution solution of 0 M to 2 M sodium chloride was flowed with a linear concentration gradient, the eluted solution was collected, and then the vaccine virus content was measured with TCID₅₀, and the impurity content was measured.

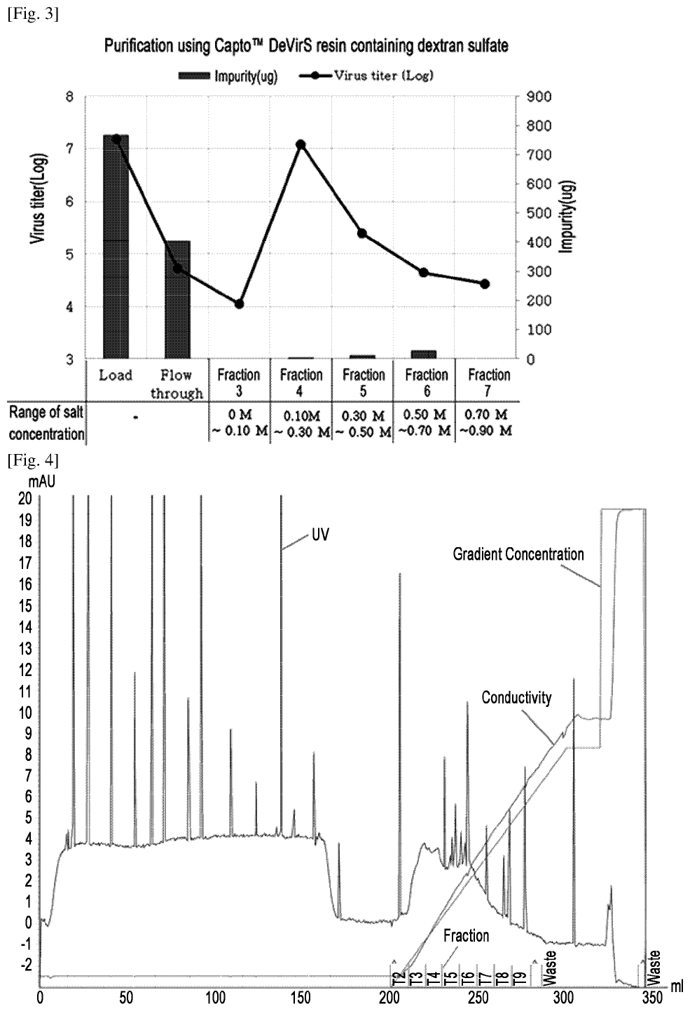

FIGS. 2 and 3 illustrate a result of purifying a vaccine virus using a Capto™ DeVirS resin containing dextran sulfate.

Referring to FIGS. 2 and 3, it was confirmed that a large amount of impurities was removed from the loaded sample when the impurity amount of the loaded sample was compared with that of flowthrough (F/T). Further, it was confirmed that most of the vaccine virus was purified without a loss of the vaccine virus when the virus content included in the loaded sample was compared with that of flowthrough (F/T).

Meanwhile, a sodium chloride concentration of the elution solution was increased from 0 M to 2 M to take respective fractions. As a result, when the fraction was taken within a salt concentration of 0.1 M to 0.9 M, preferably 0.1 M to 0.5 M, it was confirmed that a large amount of impurities was removed, and simultaneously, most of the vaccine virus was purified without a loss of the vaccine virus.

As an example, when fractions 3 and 4 were taken within a salt concentration of 0.1 M to 0.5 M, it was confirmed that the vaccine virus was recovered at about 81.1%, and at this time, the removal rate of impurities was about 97.7%, and thus the content of impurities was very low compared with other fractions.

EXAMPLE 2

### Purification using HiTrap Heparin Resin Containing Heparin

In Example 2, a purification yield for a vaccine virus and an impurity removal rate were confirmed using a HiTrap Heparin resin containing a heparin ligand.

A 50 mM Tris-HCl pH 8.0 buffer was used as an equilibrium solution and a washing solution, and an elution solution was prepared and used so that sodium chloride would reach 2 M in the equilibrium solution.

First, a vaccine virus-containing sample containing an enterovirus was loaded on a column, and then washing was performed by flowing with the washing solution. Next, an elution solution of 0 M to 2 M sodium chloride was flowed with a linear concentration gradient, the eluted solution was collected, and then the vaccine virus content was measured with TCID₅₀, and the impurity content was measured.

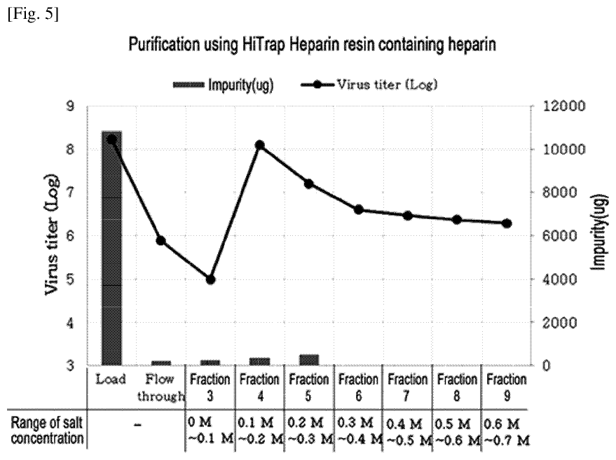

FIGS. 4 and 5 illustrate a result of purifying a vaccine virus using a HiTrap Heparin resin containing heparin.

Referring to FIGS. 4 and 5, it was confirmed that a large amount of impurities was removed from the loaded sample when the impurity amount of the loaded sample was compared with that of flowthrough (F/T). Further, it was confirmed that most of the vaccine virus was purified without a loss of the vaccine virus when the virus content included in the loaded sample was compared with that of flowthrough (F/T).

Meanwhile, a sodium chloride concentration of the elution solution was increased from 0 M to 2 M to take respective fractions. As a result, when the fraction was taken within a salt concentration of 0.1 M to 0.9 M, preferably 0.1 M to 0.5 M, and most preferably 0.1 M to 0.3 M, it was confirmed that a large amount of impurities was removed, and simultaneously, most of the vaccine virus was purified without a loss of the vaccine virus.

As an example, when fractions 4 to 7 were taken within a salt concentration of 0.1 M to 0.5 M, it was confirmed that the vaccine virus was recovered at about 85.4%, and at this time, the removal rate of impurities was 92.0%.

### Comparative Example 1

### Purification using Fractogel DEAE Resin

In Comparative Example 1, a purification yield for a vaccine virus and an impurity removal rate were confirmed using a Fractogel DEAE resin containing diethylaminoethyl (DEAE).

A 50 mM Tris-HCl pH 8.0 buffer was used as an equilibrium solution and a washing solution, and an elution solution was prepared and used so that sodium chloride would reach 2 M in the equilibrium solution.

First, a vaccine virus-containing sample containing an enterovirus was loaded on a column, and then washing was performed by flowing with the washing solution. Next, the elution solution was flowed with a linear concentration gradient, the eluted solution was collected, and then the vaccine virus content was measured with TCID₅₀, and the impurity content was measured.

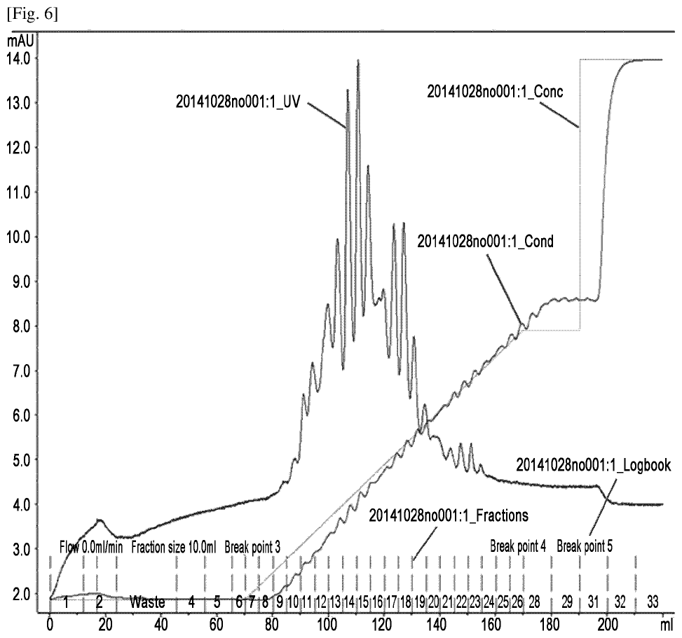

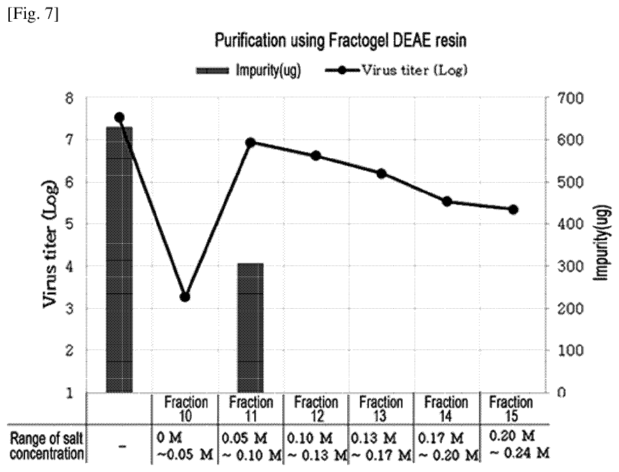

FIGS. 6 and 7 illustrate a result of purifying a vaccine virus using a Fractogel DEAE resin.

Referring to FIGS. 6 and 7, when the salt concentration was increased and respective fractions were taken, in fraction 11 at a specific salt concentration, the vaccine virus was recovered at about 25.7%, and at this time, an impurity removal rate was 51.3%. That is, it was confirmed that the impurity content was very high in a fraction in which a recovery rate of the vaccine virus was relatively high compared with other fractions.

### Comparative Example 2

### Purification using Fractogel TMAE Resin

In Comparative Example 2, a purification yield for a vaccine virus and an impurity removal rate were confirmed using a Fractogel TMAE resin containing trimethylammoniumethyl (TMAE).

A 50 mM Tris-HCl pH 8.0 buffer was used as an equilibrium solution and a washing solution, and an elution solution was prepared and used so that sodium chloride would reach 2 M in the equilibrium solution.

First, a vaccine virus-containing sample containing an enterovirus was loaded on a column, and then washing was performed by flowing with the washing solution. The elution solution was flowed with a linear concentration gradient, the eluted solution was collected, and then the vaccine virus content was measured with TCID₅₀, and the impurity content was measured.

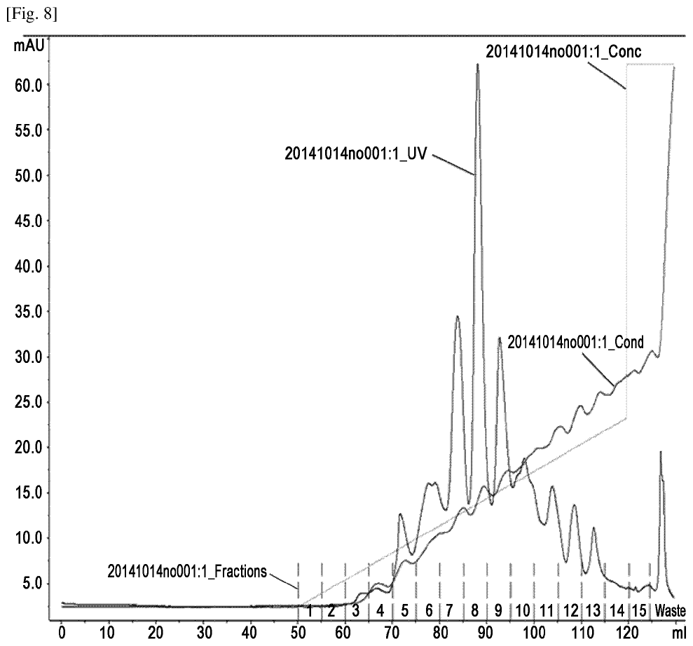

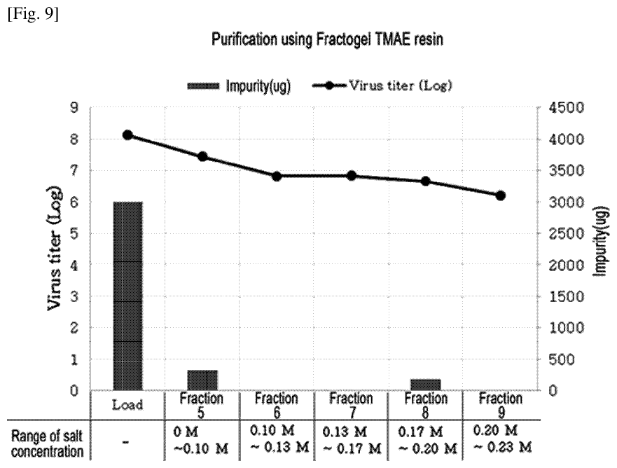

FIGS. 8 and 9 illustrate a result of purifying a vaccine virus using a Fractogel TMAE resin.

Referring to FIGS. 8 and 9, when the salt concentration was increased and respective fractions were eluted, it was confirmed that in a specific fraction (fraction 5), the vaccine virus was recovered at about 20.5%, and at this time, an impurity removal rate was 89.2%.

### Comparative Example 3

### Purification using CIM DEAE Resin

In Comparative Example 3, a purification yield for a vaccine virus and an impurity removal rate were confirmed using a disk-shaped single body consisting of DEAE.

A 50 mM Tris-HCl pH 8.0 buffer was used as an equilibrium solution and a washing solution, and an elution solution was prepared and used so that sodium chloride would reach 2 M in the equilibrium solution.

First, a vaccine virus-containing sample containing an enterovirus was loaded on a column, and then washing was performed by flowing with the washing solution. The equilibrium solution and the elution solution were mixed at predetermined ratios to flow at a concentration gradient so that a concentration of sodium chloride was 100 mM, 140 mM, 200 mM, 400 mM, and 600 mM, the eluted solution was collected, and then the vaccine virus content was measured with TCID₅₀.

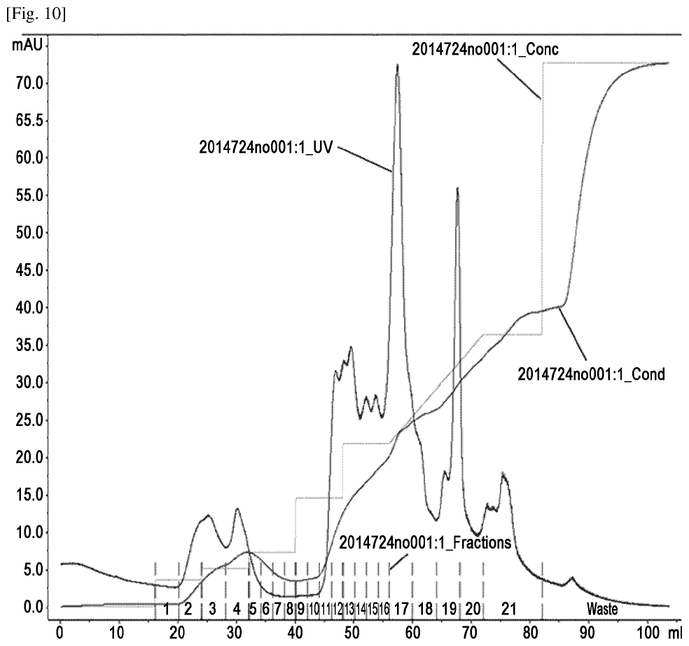

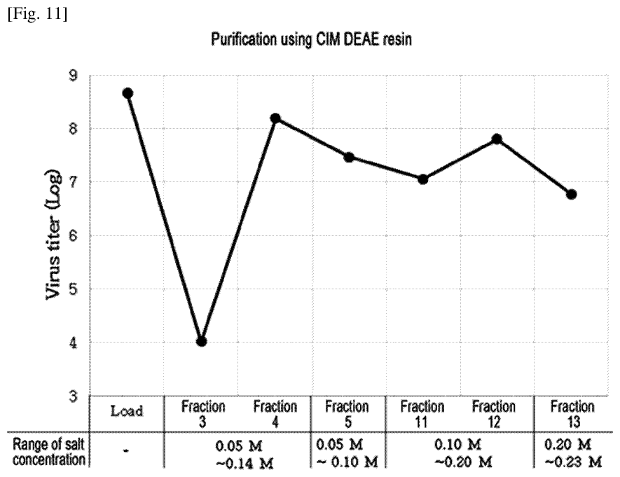

FIGS. 10 and 11 illustrate a result of purifying a vaccine virus using a CIM DEAE resin.

Referring to FIGS. 10 and 11, when the salt concentration was increased and respective fractions were eluted, it was confirmed that at a salt concentration of 140 mM, the vaccine virus was recovered at about 34.2%, and it was confirmed that application to an actual process was difficult due to overly high pressure in the process.

The methods and the results of Examples 1 and 2 and Comparative Examples 1 to 3 were summarized in Table 1 below.

TABLE 1

| | | | Comparative | Comparative | Comparative |
|---|---|---|---|---|---|
| | Example 1 | Example 2 | Example 1 | Example 2 | Example 3 |
| Resin | Capto | HiTrap | Fractogel | Fractogel | CIM DEAE |
| | DeVirS | Heparin | DEAE | TMAE | |
| Manufacturer | GE | GE | Merck | Merck | BIA |
| | | | Millipore | Millipore | separation |
| Column | 20 | 5 | 5 | 5 | 0.34 |
| volume (mL) | | | | | |
| Equilibrium | 20 mM | 50 mM | 50 mM Tris- | 50 mM Tris- | 50 mM Tris- |
| solution | Sodium | Tris-HCl | HCl pH 8.0 | HCl pH 8.0 | HCl pH 8.0 |
| | phosphate | pH 8.0 | | | |
| | pH 7.5 | | | | |
| Washing | 20 mM | 50 mM | 50 mM Tris- | 50 mM Tris- | 50 mM Tris- |
| solution | Sodium | Tris-HCl | HCl pH 8.0 | HCl pH 8.0 | HCl pH 8.0 |
| | phosphate | pH 8.0 | | | |
| | pH 7.5 | | | | |
| Elution | 20 mM | 50 mM | 50 mM Tris- | 50 mM Tris- | 50 mM Tris- |
| solution | Sodium | Tris-HCl | HCl pH 8.0 | HCl pH 8.0 | HCl pH 8.0 |
| phosphate | pH 8.0 | 0.05M to | 0M to | 0.05M to | |
| | pH 7.5 | 0.1M to | 0.1M NaCl | 0.1M NaCl | 0.14M NaCl |
| | 0.1M to | 0.5M | | | |
| | 0.5M | NaCl | | | |
| | NaCl | | | | |
| Impurity | 97.9 | 92.0 | 51.3 | 89.2 | |
| removal rate | | | | | |
| (%) | | | | | |
| Purification | 81.1 | 85.4 | 25.7 | 20.5 | 34.2 |
| yield (TCID50, | | | | | |
| %) | | | | | |

| | | | | |
|---|---|---|---|---|
| Results | Impurity removal rate | Desired | Impurity | Difficulty |
| | is very good; desired | material may | removal rate | exists in usage |
| | material may be | be separated | is very good, | due to high |
| | separated with high | with high | but pur- | pressure in |
| | yield(FIGS. 2 to 5) | yield, but | ification | process |
| | | impurity | yield is very | (FIGS. 10 and |
| | | removal rate | low | 11) |
| | | is low | (FIGS. 8 | |
| | | (FIGS. 6 and | and 9) | |
| | | 7) | | |

These results indicate that in the purification method for the vaccine virus using the affinity chromatography of the present disclosure, the desired vaccine virus may be separated with a high impurity removal rate and a high yield as compared with a conventional purification method using an ion-exchange chromatography.

It will be appreciated by those skilled in the art that the present disclosure as described above may be implemented in other specific forms without departing from the technical spirit or essential characteristics thereof. Thus, it is to be appreciated that embodiments described above are intended to be illustrative in every sense, and not restrictive. As an example, in Examples 1 and 2 described above, a purification yield of the vaccine virus and an impurity removal rate were confirmed using the resin containing dextran sulfate and the resin containing heparin, but according to Examples, a resin in which dextran sulfate and heparin are mixed at a predetermined ratio may be used.

The scope of the present disclosure is represented by the claims described below rather than the detailed description, and it is to be interpreted that the meaning and scope of the claims and all changes or modified forms derived from equivalents thereof come within the scope of the present disclosure.

1. A purification method for a vaccine virus comprising steps of:
(a) loading a sample comprising an enterovirus on an affinity chromatography column comprising a virus-affinity resin;
(b) washing the affinity chromatography column with a washing solution; and
(c) recovering a desired enterovirus from the affinity chromatography column using an elution solution.

2. The purification method of claim 1,
wherein the resin is provided to specifically bind to the enterovirus.

3. The purification method of claim 1,
wherein the resin comprises dextran sulfate.

4. The purification method of claim 1,
wherein the resin comprises heparin.

5. The purification method of claim 1,
wherein the elution solution in step (c) comprises sodium chloride.

6. The purification method of claim 1,
wherein step (c) comprises recovering a desired vaccine virus from the affinity chromatography column using an elution solution at a salt concentration of 0.1 M to 0.9 M.

7. The purification method of claim 1,
wherein step (c) comprises recovering a desired vaccine virus from the affinity chromatography column using an elution solution at a salt concentration of 0.1 M to 0.5 M.

8. The purification method of claim 1,
wherein the washing solution in step (b) comprises at least one salt selected from the group consisting of sodium phosphate, sodium chloride, Tris-HCl, 2-(N-morpholino)ethanesulfonic acid (MES), 3-morpholinopropane-1-sulfonic acid (MOPS), PIPES, potassium phosphate, potassium chloride, and 2-[4-(2-hydroxyethyl)piperazin-1-yl]ethanesulfonic acid (HEPES).

9. The purification method of claim 1, further comprising:
equilibrating the column with an equilibrium solution before step (a).

10. The purification method of claim 9,
wherein the equilibrium solution comprises at least one salt selected from the group consisting of sodium phosphate, sodium chloride, Tris-HCl, 2-(N-morpholino)ethanesulfonic acid (MES), 3-morpholinopropane-1-sulfonic acid (MOPS), PIPES, potassium phosphate, potassium chloride, and 2-[4-(2-hydroxyethyl)piperazin-1-yl]ethanesulfonic acid (HEPES).

11. The purification method of claim 1, further comprising:
equilibrating the column with an equilibrium solution after at least one of steps (a) and (b).

12. The purification method of claim 1, further comprising:
re-equilibrating the column with a re-equilibrium solution after at least one of steps (a) and (b).

13. The purification method of claim 1,
wherein the sample is prepared from host cells other than human-derived cells.
